(19) United States
(12) Patent Application Publication
(10) Pub. No.: US 2005/0234529 A1
(43) Pub. Date: Oct. 20, 2005

(54) Method and apparatus for tuning of the cochlea

(75) Inventors: John S. Oghalai, Pearland, TX (US); Bahman Anvari, Houston, TX (US)

Correspondence Address: CONLEY ROSE, P.C., P. O. BOX 3267, HOUSTON, TX, 77253-3267, US

(73) Assignees: Baylor College of Medicine, Houston, TX (US); William Marsh Rice University, Houston, TX (US)

(21) Appl. No.: 11/082,574

(22) Filed: Mar. 17, 2005

Related U.S. Application Data
(60) Provisional application No. 60/554,160, filed on Mar. 18, 2004.

(51) Int. Cl. A61N 5/067

(52) U.S. Cl. 607/89

(57) ABSTRACT

A method and apparatus are disclosed for improving hearing by irradiating the cochlea with a laser beam. In one embodiment, an apparatus for improving hearing comprises a laser source that emits at least one laser beam to irradiate at least a portion of a basilar membrane. The laser source emits the at least one laser beam under suitable conditions to change a frequency response of the basilar membrane. In an embodiment, the laser source irradiates the cochlea at a defined pulse duration to change the physical properties of the cochlea. The irradiated portion of the cochlea may be the basilar membrane.

### CROSS-REFERENCE TO RELATED APPLICATIONS

This non-provisional application claims the benefit of U.S. Provisional Application No. 60/554,160, filed Mar. 18, 2004, which is hereby incorporated by reference in its entirety.

### STATEMENT REGARDING FEDERALLY SPONSORED RESEARCH OR DEVELOPMENT

This work was supported by N1H-NIDCD grant number DC05 131.

### BACKGROUND OF THE INVENTION

1. Field of the Invention

This invention relates to the field of hearing improvement and more specifically to the field of tuning the cochlea to improve hearing.

2. Background of the Invention

Hearing loss (e.g., sensorineural hearing loss) in a subject such as a human or animal may have many different causes. For instance, a common cause of hearing loss is presbyacusis, which typically occurs with aging. Presbyacusis may be a result of chronic low intensity noise exposure as well as genetic factors. Other common causes of hearing loss may include high intensity noise exposure, toxicity, and genetic predisposition.

The typical patient with sensorineural hearing loss may have elevated thresholds in the high frequencies, with low frequency thresholds closer to or within the normal range. In regards to hearing loss due to noise exposure, such elevated thresholds in the high frequencies may include morphological changes in outer hair cells. Morphological changes in outer hair cells may include loss of the cochlear amplifier in the high frequency range of the cochlea. Sensorineural hearing loss is typically treated by mechanical hearing aids, which operate by compressing and amplifying incoming sounds. Drawbacks to mechanical hearing aids include the mechanical hearing aid not compensating for loss of the cochlear amplifier because the mechanical hearing aid does not improve frequency discrimination.

Consequently, there is a need for an apparatus that can restore hearing loss. Additional needs include an apparatus that can improve hearing. Further needs include a method for improving hearing.

### BRIEF SUMMARY OF SOME OF THE PREFERRED EMBODIMENTS

These and other needs in the art are addressed in one embodiment by an apparatus for improving hearing. The apparatus comprises a laser source that emits at least one laser beam to irradiate at least a portion of a basilar membrane.

In another embodiment, these and other needs in the art are addressed by an apparatus for improving hearing. The apparatus comprises a laser source that emits at least one laser beam into at least a portion of an inner ear to change a frequency response of the inner ear.

A further embodiment addresses these and other needs in the art by a method for changing the frequency response of a cochlea. The method comprises introducing a laser source into an inner ear, wherein the cochlea is disposed within the inner ear. In addition, the method comprises emitting at least one laser beam from the laser source. The method further comprises irradiating at least a portion of the cochlea to change a physical property of the cochlea to change the frequency response.

An apparatus that improves hearing by using a laser to change the frequency response of the basilar membrane overcomes problems in the art. For instance, the apparatus may improve the ability of the cochlea to amplify sounds (e.g., the cochlear amplifier). In addition, the basilar membrane itself is affected by the apparatus to improve hearing rather than solely a reliance on a mechanical device to amplify sound.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates the organ of Corti and the basilar membrane;

FIG. 2 illustrates an exploded view of the basilar membrane;

FIG. 3 illustrates a cochlea tuning apparatus having a laser source and a delivery device;

FIG. 4 illustrates an example showing quantification of the effects of laser irradiation on different areas of the cochlea;

FIG. 5 illustrates an example showing intensity of birefringence for different laser pulses;

FIG. 6 illustrates an example of basilar membrane velocity;

FIG. 7 illustrates an example of basilar membrane frequency;

FIG. 8 illustrates birefringence for an acute sample;

FIG. 9 illustrates ABR thresholds for an acute sample;

FIG. 10 illustrates birefringence for a delayed sample; and

FIG. 11 illustrates ABR thresholds for a delayed sample.

### DETAILED DESCRIPTION OF THE PREFERRED EMBODIMENTS

In an embodiment, hearing may be improved in a subject by changing physical properties of the basilar membrane of the subject, which may be accomplished by a cochlea tuning apparatus comprising a laser source. Without being limited by theory, changing physical properties such as mass and stiffness of the basilar membrane causes a shift in the frequency response of the cochlea, which may result in an improvement in high frequency hearing. It is to be understood that the subject to which the cochlea tuning apparatus may be utilized may include any suitable subjects such as humans or other mammals.

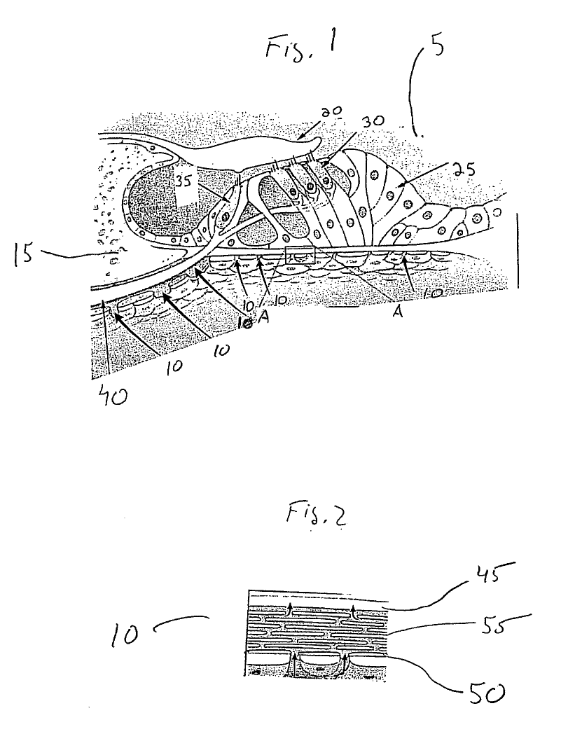

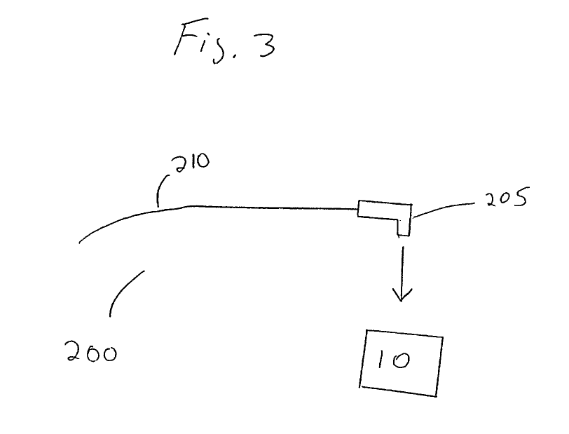

The cochlea is a portion of the inner ear in which sound pressure waves are converted into electrical signals and passed to the brain. The cochlea includes the basilar membrane and the organ of Corti. The basilar membrane refers to a membranous portion of the cochlea in the inner ear that provides support to the organ of Corti. The basilar membrane may comprise collagen fibers, proteoglycans, glycosaminoglycans, and/or adhesive proteins such as fibronectin and laminin. The organ of Corti refers to a structure that is located in the cochlea on the inner surface of the basilar membrane and that contains hair cells for transducing sound vibrations into electrical energy that is transmitted to nerve fibers. FIG. 1 illustrates the organ of Corti 5, basilar membrane 10, osseus spiral lamina 15, and tectorial membrane 20. Organ of Corti 5 comprises supporting cells 25, outer hair cells 30, and inner hair cells 35. Auditory nerve 40 is also shown, which transmits the electrical signals to the brain. FIG. 2 illustrates an exploded view of basilar membrane 10 as shown in FIG. 1 at section A-A. In FIG. 2, it can be seen that basilar membrane 10 comprises an upper portion 45 and a lower portion 50. Collagen fibers 55 may be organized between upper portion 45 and lower portion 50. It is to be understood that sound pressure waves from the environment enter the ear and form sound pressure waves within the fluids of the inner ear comprising the cochlea, in which the sound pressure waves may be transduced into electrical signals that are then passed to the brain via auditory nerve 40.

The cochlea tuning apparatus comprises a laser source and a delivery device. The laser source may comprise any suitable source for emitting a laser beam in the cochlea. Without limitation, examples of suitable laser sources include gas lasers, excimer lasers, dye lasers, solid state lasers, and ultrafast lasers. It is to be understood that the type of laser source may be chosen based upon factors such as, without limitation, tissue composition, optical properties, the portion of the basilar membrane to be irradiated, and the like. Ultrafast lasers refer to lasers having pulse durations in the femtosecond to nanosecond ranges. In an embodiment, an exogenous chromophore such as a dye may be used to localize and enhance laser light absorption to and by the basilar membrane. In another embodiment, the lasers may have any wavelength that may be suitable for exciting a chromophore in the cochlea. For instance, the laser may have a wavelength that reacts with a chosen chromophore but that has little or no reaction with tissue in the cochlea that is not stained by the chromophore, alternatively the laser has a wavelength from about 300 nm to about 10 μm. The laser may also have any pulse duration suitable for changing physical properties of the basilar membrane. In an embodiment, the laser may have a pulse duration suitable to photocoagulate a portion of the basilar membrane, alternatively from about 0.01 microseconds to about 1 second, and alternatively from about 0.1 microseconds to about 100 milliseconds. In another embodiment, the laser may have pulse durations of at least about 1 femtosecond, alternatively from about 1 femtosecond to about 100 nanoseconds, and alternatively from about 10 femtoseconds to about 100 femtoseconds.

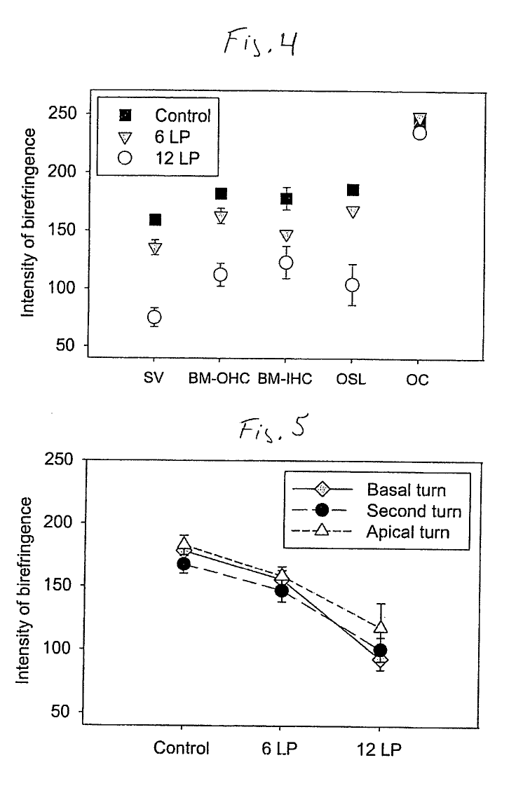

The delivery device may be any suitable device for directing the laser source to a suitable location at which the laser source may emit a laser beam and irradiate at least a portion of the basilar membrane. Without limitation, examples of suitable delivery devices include a rod, cable, optical fiber, endoscope, and the like. The delivery device may be flexible or substantially rigid. It is to be understood that characteristics of the delivery device such as its length, width, and flexibility may be selected based upon factors such as the type of laser source, the method used for applying the cochlea tuning apparatus, and the like. For example, as illustrated in FIG. 3, an embodiment of the cochlea tuning apparatus 200 may include a laser source 205 secured to an end of a flexible fiberoptic cable 210. It is to be understood that the laser source may be attached to the delivery device (e.g., fiberoptic cable 210) by any suitable means such as clipping, clamping, soldering, adhesives, press fit, and the like. In an embodiment, the delivery device may include optics at its distal end to focus the light emitted from the source.

In an embodiment, the cochlea tuning apparatus may further comprise a spacer unit, which may facilitate directing the laser beam at the basilar membrane. The spacer unit may include a distance measuring device, which is suitable for indicating the distance from the laser source to the cochlea. The distance measuring device is suitable for use in the inner ear and may be attached to the cochlea tuning apparatus. The distance measuring device may indicate to the operator the distance of the cochlea to the cochlea tuning apparatus, which may allow the operator to control the distance of the laser source to the cochlea. Without limitation, such a device may include an infrared distance measuring device, a laser measuring device, a mechanical measuring device, and the like. In some embodiments, the spacer unit may also include a positioning device that is suitable for positioning the cochlea tuning apparatus within the ear canal. For instance, the positioning device may center the cochlea tuning apparatus within the ear canal.

In an embodiment, the basilar membrane of the cochlea may be identified, and the cochlea tuning apparatus may be introduced into the cochlea of the inner ear in which the cochlea tuning apparatus emits a laser beam. In an alternative embodiment, more than one laser beam may be emitted. The cochlea tuning apparatus changes a physical property of the basilar membrane by irradiation. Changing a physical property may include decreasing the mass of the basilar membrane, increasing the stiffness of the basilar membrane, or combinations thereof. Stiffness refers to rigidity of the basilar membrane. Without being limited by theory, the basilar membrane may be tonotopically tuned so that high frequencies may be represented at the lower portion of the basilar membrane (e.g., lower portion 50 of FIG. 2), and low frequencies may be represented at the upper portion of the basilar membrane (e.g., upper portion 45 of FIG. 2). Decreasing the mass and/or increasing the stiffness of the basilar membrane may cause a shift in the frequency response of the cochlea and thereby transform a low frequency region of the basilar membrane (e.g., upper portion 45) to be responsive at a higher frequency. In an embodiment, stiffness of the basilar membrane may increase from about 1 percent to about 200 percent the normal stiffness of the basilar membrane. The normal stiffness of the basilar membrane refers to the stiffness of the basilar membrane before exposure to the laser beam.

The cochlea tuning apparatus may be introduced into the ear by any suitable method. Without limitation, the cochlea tuning apparatus may be introduced into the ear through an incision and/or through a cavity in the body of the subject. Introducing the cochlea tuning apparatus through an incision includes making an incision in the inner ear of a size suitable for entry of the laser source into the inner ear. The laser may be introduced through an incision made in the eardrum, by lifting up the eardrum, or through a post-auricular incision and mastoidectomy approach. The laser may be aimed at the basilar membrane either through the promontory of the middle ear, through the round window membrane, or by threading a thin fiberoptic cable up inside the cochlea. In an embodiment, the delivery device may direct the laser source to a suitable location in the inner ear to irradiate the basilar membrane.

In an embodiment, a chromophore may be inserted into the cochlea to stain a portion or substantially all of the basilar membrane. Without being limited by theory, the chromophore absorbs light from the laser source and becomes excited, which may change the conformation of the collagen proteins within the stained portion of the basilar membrane. By changing the conformation of the collagen proteins, the irradiated portion of the basilar membrane may have increased stiffness. The duration at which the chromophore may be irradiated may be selected based upon factors such as the type of laser source, duration of laser pulses, type of chromophore, and how much of the basilar membrane may be stained by the chromophore. In an embodiment, the irradiation may be of a duration from about 0.01 microseconds to about 1 millisecond, alternatively from about 1 millisecond to about 1 second. A chromophore refers to a chemical group that has selective light absorption. In an embodiment, the chromophore is an exogenous chromophore. Without limitation, an example of a suitable chromophore is a dye. It is to be understood that a chromophore may be chosen that is suitable for the type of laser source based upon factors such as laser wavelength and its binding properties. In an embodiment, the laser source is a dye laser.

The chromophore may be inserted into the cochlea and applied to stain the basilar membrane by any suitable method. Without limitation, examples of suitable methods include using an infusion pump, a pipette, a syringe, diffusion through the round window membrane after transtympanic application, and after systemic intravenous injection. For instance, a syringe may be inserted into the ear and a sufficient amount of chromophore may be introduced to the cochlea to stain at least a portion of the cochlea with substantially all or a portion of the basilar membrane being stained. In an alternative embodiment, a portion of the basilar membrane is stained by the chromophore. In such an alternative embodiment, only a small amount of dye may be locally applied to the location desired as the target. In another alternative embodiment, a dye may be selected that binds only to certain areas or structures within the basilar membrane, which may include the use of an antibody. A laser source may emit a laser beam and irradiate substantially all of the cochlea (including substantially all of the basilar membrane) or irradiate a portion of the basilar membrane. Without being limited by theory, the chromophore attracts the laser beam from the laser source and may therefore reduce exposure to the laser beam of the non-basilar membrane portion of the cochlea.

In another embodiment, a primary antibody may be introduced to the cochlea. The primary antibody may be an antibody that within the cochlea only reacts with the basilar membrane. An antibody refers to a protein that specifically binds with certain other substances. Without limitation, examples of suitable primary antibodies include chicken anti-fibronectin antibodies. A secondary antibody with an attached chromophore may then be introduced to the cochlea. The secondary antibody may attach to the primary antibody. A secondary antibody may include any antibody that may attach to the primary antibody and have an attached chromophore. Without limitation, examples of a suitable secondary antibody includes goat anti-chicken antibodies. The laser source may irradiate a portion or substantially all of the basilar membrane to excite the chromophore and increase the stiffness of the basilar membrane.

In another embodiment, the cochlea tuning apparatus irradiates the basilar membrane to provide a photochemical reaction. In this embodiment, the exogenous chromophore absorbs the radiation and undergoes chemical reactions (e.g., production of free radicals) that result in oxidation (e.g., necrosis) of the targeted tissue. In such an embodiment, no heat is generated. In this embodiment, the laser source may have a pulse duration from about 1 second to about 10 minutes.

In an alternative embodiment, the cochlea tuning apparatus irradiates the cochlea (e.g., basilar membrane) without the presence of an exogenous chromophore. In such an alternative embodiment, the laser irradiates the basilar membrane for a sufficient duration in which the basilar membrane absorbs light from the laser source and becomes excited, which may change the conformation of the collagen proteins. The duration at which the basilar membrane may be irradiated may be selected based upon factors such as the type of laser source, duration of laser pulses, the degree of change needed to obtain the desired effect, and basilar membrane thickness. In an embodiment, the irradiation may be of a duration from about 0.1 microseconds to about 1 microsecond, alternatively from about 1 microsecond to about 1 millisecond.

It is to be understood that the cochlea tuning apparatus is not limited to using dye lasers when chromophores are placed in the cochlea. In alternative embodiments, other lasers such as an ultrafast laser may be used without exogenous chromophores to irradiate the basilar membrane. For instance, an ultrafast laser may be used to photoablate the basilar membrane to reduce its mass.

In another embodiment, the laser source is an ultrafast laser that may irradiate the basilar membrane to change its mass without the use of chromophores. In such an embodiment, the laser source is directed to a suitable location in the cochlea at which the laser source may direct laser energy underneath the basilar membrane to change its mass. Without being limited by theory, the ultrafast laser pulse directly induces intermolecular ruptures in the basilar membrane, which may result in a conformation of the collagen proteins and/or mass removal. In an embodiment, the laser source is focused to irradiate only the basilar membrane, which produces plasma that may only substantially affect the basilar membrane. In this embodiment, the ultrafast laser has a pulse range from about 1 femtosecond to about 100 nanoseconds, and alternatively from about 10 femtoseconds to about 100 femtoseconds.

In an embodiment, the cochlea tuning apparatus ablates at least a portion of the basilar membrane. In such an embodiment, the cochlea tuning apparatus has a laser source comprising an ultrafast laser. The ultrafast laser may remove any desired portion of the basilar membrane to achieve the desired increase in high frequency response. In an embodiment, the ultrafast laser removes from about 0.001 microns to about 5 microns of the basilar membrane, alternatively from about 0.01 microns to about 5 microns, and alternatively from about 0.001 microns to about 1 micron, and further alternatively from about 0.1 microns to about 1 micron. In an embodiment, the delivery device introduces the ultrafast laser to an appropriate location in the cochlea at which only the basilar membrane is exposed to the laser beam emitted from the ultrafast laser. Without being limited by theory, irradiating only the basilar membrane with the ultrafast laser may reduce collateral damage (e.g., damage to non basilar membrane tissue in the cochlea). In an embodiment, ablation may be achieved by plasma-mediated ionization. Plasma-mediated ionization involves removing electrons from the basilar membrane by ionizing the basilar membrane. For instance, high intensity laser pulses from an ultrafast laser may have sufficient energy to detach electrons from the core of the targeted material. High intensity laser pulses refer to pulses of from about 10⁷ W/cm² to about 10¹⁵ W/cm².

It is to be understood that the cochlea tuning apparatus is not limited to comprising a laser source and a delivery device but in alternative embodiments may comprise the laser source without a delivery device. For instance, the laser source may be of a size suitable for insertion into the inner ear without a delivery device. In other instances, the cochlea may be removed from the subject and irradiated by the laser source outside of the body of the subject. After suitable irradiation by the laser source and adjustment of a physical property of the basilar membrane, the cochlea may be implanted back into the inner ear. In other alternative embodiments, the cochlea tuning apparatus comprises non-laser sources that heat the basilar membrane to provide a change in the frequency response. For instance, examples of suitable non-laser sources include ultrasound sources, microwave sources, radio frequency sources, and heated contact probes.

To further illustrate various illustrative embodiments of the present invention, the following examples are provided.

### EXAMPLE 1

In Example 1, the cochlea of guinea pigs were stained with a chromophore and irradiated with laser beams. The guinea pigs were sacrificed by guillotine. Their cochleae were excised from the tympanic bulla and placed into a Petri dish filled with artificial perilymph (solution of in mM:130 NaCl, 4 HCl, 1 MgCl2, 2 CaCl2, 10 HEPES, 10 Glucose). The pH was titrated to 7.3, and the osmolarity was 293 mOsm.

The membranous structures within the cochlea were transparent to visible laser irradiation and were bathed in the aqueous artificial perilymph environment. To achieve optical selectivity for the basilar membrane and to enhance laser energy absorption, trypan blue was used as an exogenous chromophore. Trypan blue is a water soluble stain with a maximum absorption at 607 nm. The excised cochlea was perfused with 0.3 ml of 0.1% trypan blue solution dissolved in artificial perilymph. In order to perfuse the stain, an opening in the bone overlying the scala tympani of the basal turn was then created using a straight needle. Another opening was created at the apex of the cochlea, at the helicotrema. The cochlea was perfused using a flexible micropipette inserted into the opening at the base of the cochlea. The stain circulated through the scala tympani along the entire length of the cochlear duct, until it was observed to flow from the apical opening. After 3 minutes, unbound stain was washed out with 0.3 ml of artificial perilymph.

The cochlea was placed in a dry Petri dish to reproduce the air-filled middle ear cavity that exists in vivo. Due to surface tension forces, the artificial perilymph remained within the cochlea. A 600 nm pulsed dye laser (SCLEROPLUS of Candela Corp., Wayland, Mass.) was used to irradiate the cochlea. Several different laser exposure protocols were tested to illicit histological changes in the basilar membrane, from one laser pulse at radiant exposure of 5 J/cm² to 12 pulses of 30 J/cm². It was found that histological changes occurred only at the higher energy levels. A radiant exposure of 30 J/cm was used.

Laser irradiation was performed within three hours of animal sacrifice. The pulse duration was 1.5 ms, and the spot size was 5 mm. The cochleae were irradiated with either 6 or 12 laser pulses with a repetition rate of 1 Hz. The optics were positioned so that the laser beam was delivered to the undersurface of the cochlea, perpendicular to the basilar membrane. Two controls were used. One control was not irradiating the contralateral cochlea, but instead it was stained with trypan blue and washed out. The second control was that the otic capsule bone of the irradiated cochlea was not stained but was still exposed to the same amount of laser irradiation.

The cochleae were fixed in 4% paraformaldehyde immediately after laser irradiation. After two days of fixation, the cochleae were decalcified in 0.1 M EDTA for four days. The samples were then embedded in paraffin and cut in 6 μm sections. For better visualization of the tissue birefringence, the sections were stained with picrosirius red and haematoxilin and analyzed using polarized light microscopy. The control cochleae were fixed in the same manner as the irradiated cochlea.

Images were captured digitally using a 6.3 megapixel camera connected to an upright microscope. The applied light, aperture size, and time exposure were held constant for all images. ImageJ was used to quantify the intensity of birefringence within different cochlear tissues. The color image was converted to 8 bit grey scale. After conversion, areas of high birefringence were white, and areas of low birefringence were grey or black. From the regions of interest, areas with highest birefringence were chosen, and the signal intensity was determined by averaging at least 60 pixels.

It was observed that perilymphatic perfusion of the cochlea with trypan blue demonstrated residual stain within the cochlear turns after washout. The otic capsule bone did not stain. It was also observed that trypan blue predominantly stained the area of the organ of Corti and the basilar membrane. In the control cochlea, it was observed that polarized light microscopy demonstrated sites of highly organized collagen. The collagen within the otic capsule bone surrounding the cochlea had a normal trabecular pattern. The collagen within the osseous spiral lamina and the spiral ligament converged towards the insertion of the basilar membrane. In addition, the basilar membrane demonstrated strong birefringence in cross-section. This reflected the parallel distribution of collagen fibers. It was also observed that laser irradiation diminished the natural birefringence associated with the collagen organization of the basilar membrane.

Quantification of the birefringence was performed to assess dose-dependent and region dependent effects. FIG. 4 illustrates quantification of the effects of the laser irradiation. The birefringence intensity of various cochlear tissues were averaged from all three turns and plotted on a scale from 0-255 with 0 describing minimal and 255 maximal birefringence. The different tissues included the basilar membrane under the outer hair cells (BM-OHC), the basilar membrane under the inner hair cells (BM-IHC), the osseous spiral lamina (OSL), and the stria vascularis (SV). A single measurement of the otic capsule bone (OC) was plotted as a control. Birefringence was reduced with a greater number of laser pulses (LP) applied. This occurred in all of the structures stained by the exogenous chromophore, trypan blue. The otic capsule bone was unstained and did not demonstrate a reduction in its birefringence. The basilar membrane under the inner hair cells, the basilar membrane under the outer hair cells, the stria vascularis, and the osseous spiral lamina all had larger reductions in birefringence with 12 laser pulses compared to 6 laser pulses as shown in FIG. 4, which showed that all of these tissues were stained with trypan blue and that the changes in collagen had a dose-dependence. The non-stained collagen of the otic capsule bone was not affected by laser irradiation.

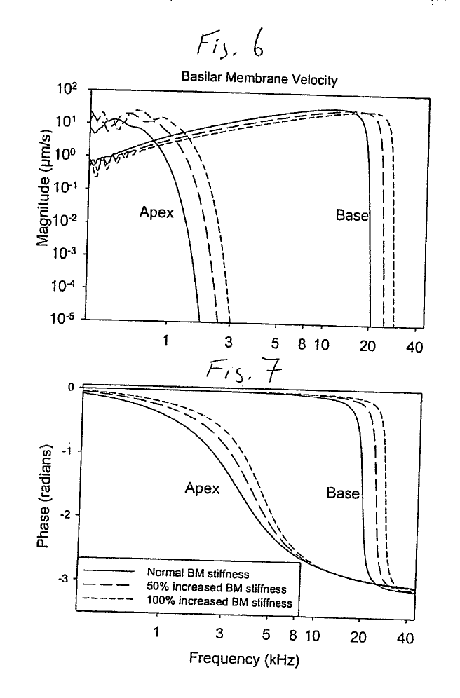

FIG. 5 further illustrates quantification of the effects of the laser irradiation. The average birefringence of the basilar membrane under the outer hair cells, the basilar membrane under the inner hair cells, the osseous spiral lamina, and the stria vascularis were plotted versus cochlear turn. Equivalent changes of the basilar membrane birefringence were observed in all turns of the cochlea. This was due to the relatively few and thin absorbing structures that permitted sufficient light penetration through the cochlea.

In all stained tissues, it was observed that the reductions in the intensity of birefringence were similar for tissues in each of the cochlear turns, which suggested that all areas of the cochlea are exposed to equal amounts of laser energy. In addition, it was further observed that the colored beam stop under the Petri dish became photobleached, even though it had to pass through all three cochlear turns and the otic capsule bone to reach it.

Because the basilar membrane is a tuned structure, changing its stiffness would modulate the resonant frequency of its velocity. In order to estimate what changes in cochlear tuning occurred, a mathematical model of passive cochlear mechanics was used. The model is taught in Choi, et al., “A cochlear model designed to test the effect of modulating outer hair cell biophysical properties on basilar membrane mechanics,” 2004, Twenty-Seventh Annual Midwinter Research Meeting of the Association of Research in Otolaryngology, which is herein incorporated by reference in its entirety. Basilar membrane velocity was calculated at two different cochlear locations, the base and the apex, in response to sine wave stimulation across the frequency spectrum, which is shown in FIGS. 6 and 7. FIGS. 6 and 7 are shown assuming three different basilar membrane stiffnesses: normal, 50% increased, and 100% increased. Increasing basilar membrane stiffness shifted the resonant frequency towards higher frequencies. Doubling basilar membrane stiffness raised the resonant frequency by a factor of 1.5 (from 1 to 1.5 kHz at the apex and from 20 to 30 kHz at the base). The pattern of the shift was similar at both the base and the apex of the cochlea.

The model suggested that the amount of frequency shift may be considerable. Laser irradiation of the cochlea may be used therapeutically. Laser irradiation caused immediate changes in collagen organization within the cochlea and within the basilar membrane, which may be visualized with polarization microscopy.

### EXAMPLE 2

In Example 2, the acute and delayed histological functional changes after in vivo cochlear laser photoirradiation of mice was studied. C57 black female mice were used that were aged 4-8 weeks. A trypan blue chromophore (0.1%) was applied through the round window. A 600 nm pulsed dye laser was used to irradiate the mice. The laser had a pulse duration of 1.5 ms and a 10 mm spot size.

ABR monitoring was accomplished by 5 msec tone pip stimuli. 250 repetitions were averaged, and the thresholds were calculated off-line.

Some animals were sacrificed immediately after laser irradiation (acute), and some animals were sacrificed after 2 weeks of incubation (delayed).

For histology, paraffin-embedded sections were stained with picrosirius red and analyzed with polarization microscopy.

Image J (NIH) was used to quantify tissue birefringence. Representative areas of the various tissues containing at least 60 pixels were averaged. Five sequential sections from each cochlea were analyzed and averaged.

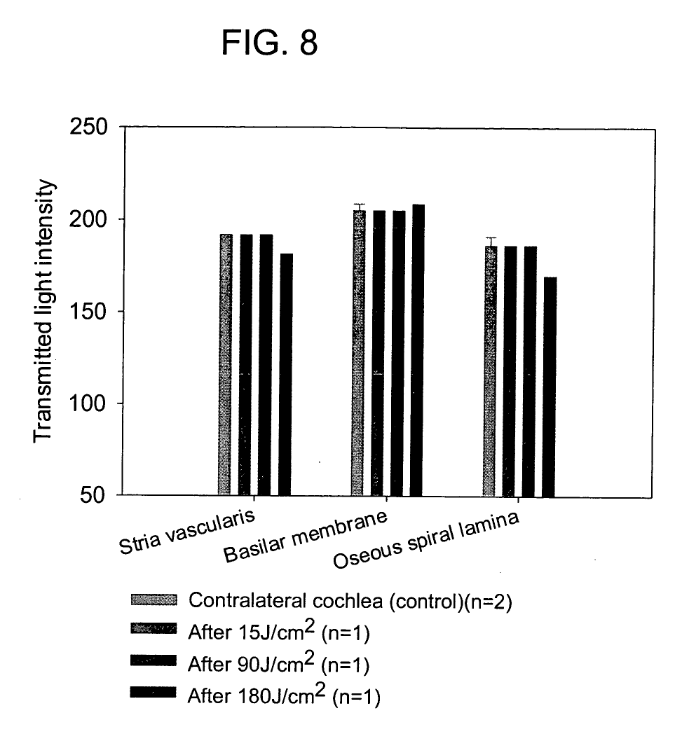

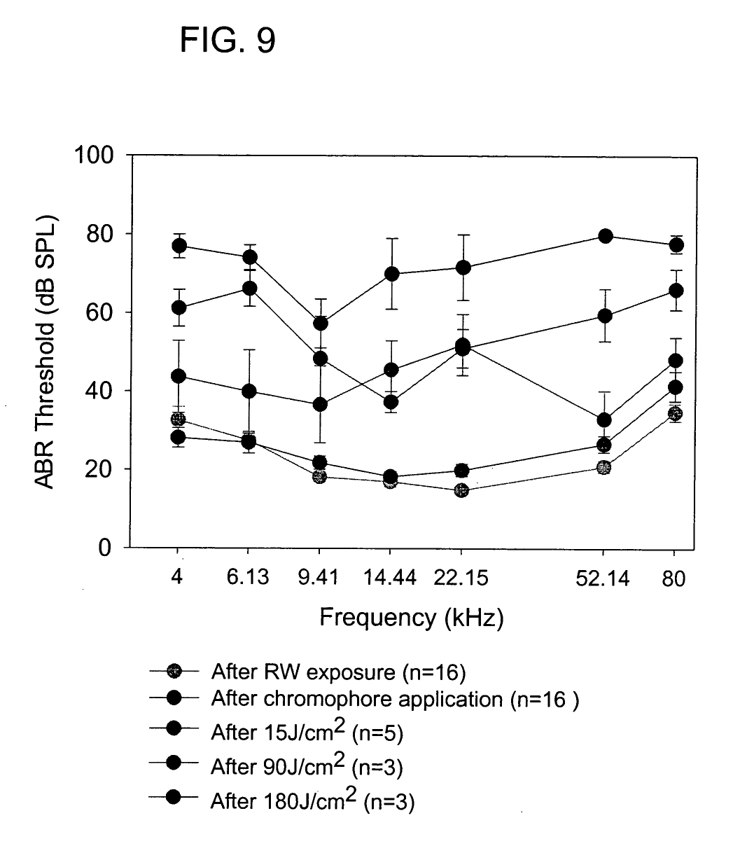

For acute effects, it was observed that polarized light micrographics of the mice cochlea irradiated with 15, 90, and 180 J/cm² had no obvious changes in birefringence. FIG. 8 illustrates quantification of the birefringence. It can be seen that quantification of the birefringence showed no changes. FIG. 9 shows ABR threshold. It can be observed from FIG. 9 that chromophore application did not cause threshold shifts but did cause dose-dependent threshold shifts.

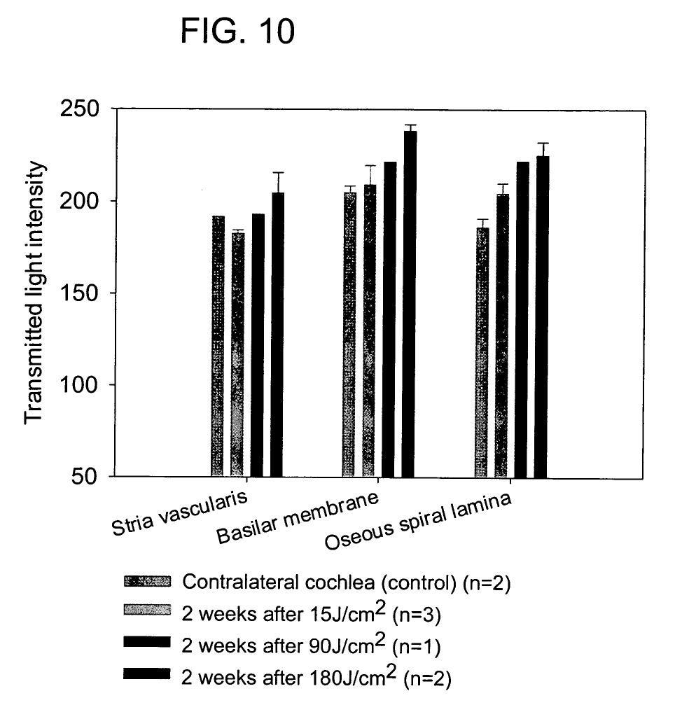

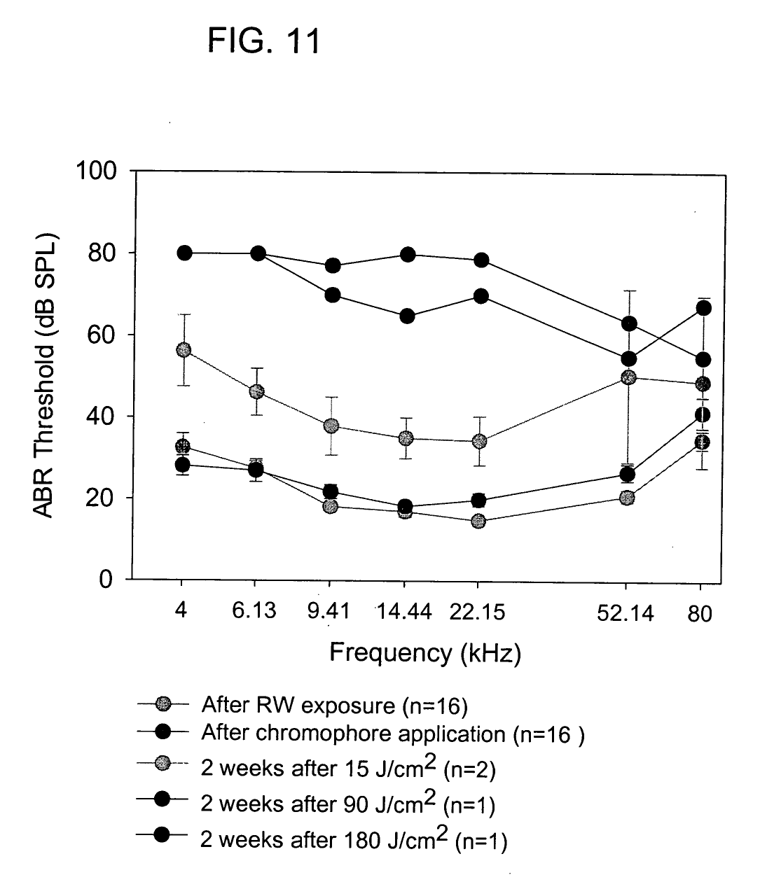

For delayed effects after two weeks incubation, it was observed that polarized light micrographics of the mice cochlea irradiated with 15, 90, and 180 J/cm² had new collagen deposition within the basilar membrane, which caused it to thicken. In addition, high laser doses caused a strong inflammatory response throughout the scala tympani and scala vestibuli. FIG. 10 illustrates quantified birefringence. It can be seen from FIG. 10 that quantification of the birefringence showed that higher laser doses led to an increase in tissue birefringence. FIG. 11 illustrates the ABR thresholds. From FIG. 11, it was also observed that the threshold shifts were similar to those of the acute effects group.

Therefore, it was observed that laser irradiation caused minimal change in collagen as measured by birefringence but may cause signification threshold elevations. In addition, after two weeks, histologic manifestations of laser photocoagulation were noted. Therefore, laser irradiation may induce a change in basilar membrane stiffness and modulate cochlear tuning.

Although the present invention and its advantages have been described in detail, it should be understood that various changes, substitutions and alterations may be made herein without departing from the spirit and scope of the invention as defined by the appended claims.

1. An apparatus for improving hearing, comprising:
a laser source that emits at least one laser beam to irradiate at least a portion of a basilar membrane.

2. The apparatus of claim 1, wherein the laser source emits the at least one laser beam under suitable conditions to change a frequency response of the basilar membrane.

3. The apparatus of claim 1, wherein the irradiation increases the stiffness of the basilar membrane.

4. The apparatus of claim 1, wherein the at least one laser beam excites a chromophore on the basilar membrane.

5. The apparatus of claim 4, wherein the chromophore is attached to a secondary antibody, and wherein the secondary antibody is attached to a primary antibody that is attached to the basilar membrane.

6. The apparatus of claim 1, wherein the laser source emits the at least one laser beam with a wavelength from about 300 nm to about 10 μm.

7. The apparatus of claim 1, wherein the irradiation decreases the mass of the basilar membrane.

8. The apparatus of claim 7, wherein the mass of the basilar membrane is decreased by the at least one laser beam removing from about 0.001 microns to about 5 microns of the basilar membrane.

9. The apparatus of claim 1, wherein the laser source comprises an ultrafast laser.

10. An apparatus for improving hearing, comprising:
a laser source that emits at least one laser beam into at least a portion of an inner ear to change a frequency response of the inner ear.

11. The apparatus of claim 10, wherein the inner ear comprises a basilar membrane, and further wherein the irradiation increases the stiffness of the basilar membrane.

12. The apparatus of claim 10, wherein the at least one laser beam excites a chromophore in the inner ear.

13. The apparatus of claim 10, wherein the irradiation decreases the mass of the basilar membrane.

14. The apparatus of claim 13, wherein the mass of the basilar membrane is decreased by the at least one laser beam removing from about 0.001 microns to about 5 microns of the basilar membrane.

15. A method for changing a cochlea frequency response, comprising:
(A) introducing a laser source into an inner ear, wherein the cochlea is disposed within the inner ear;
(B) emitting at least one laser beam from the laser source; and
(C) irradiating at least a portion of the cochlea to change a physical property of the cochlea to change the frequency response.

16. The method of claim 15, wherein step (A) further comprises introducing a chromophore into the cochlea to stain at least a portion of the cochlea.

17. The method of claim 16, wherein changing the physical property further comprises exciting the chromophore.

18. The method of claim 15, wherein step (C) further comprises ablating at least a portion of the cochlea.

19. The method of claim 18, further comprising emitting the at least one laser beam at a pulse rate of at least about 1 femtosecond.

20. The method of claim 15, wherein the laser source is introduced into the ear by an incision.
